(19) United States
(12) Patent Application Publication
(10) Pub. No.: US 2005/0203637 A1
(43) Pub. Date: Sep. 15, 2005

(54) Structures and devices for parenteral drug delivery and diagnostic sampling

(76) Inventors: Carl F. Edman, San Diego, CA (US); Darrel D. Drinan, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/984,681

(22) Filed: Nov. 8, 2004

Related U.S. Application Data
(60) Provisional application No. 60/519,060, filed on Nov. 10, 2003.

(51) Int. Cl. A61F 2/02; B23K 26/36; C25D 5/48

(52) U.S. Cl. 623/23.76; 216/56; 219/121.69; 205/223

(57) ABSTRACT

A structure for and method of manufacture of structures for implantation within the tissue of a mammalian subject are disclosed. These structures can be utilized in applications such as the delivery of therapeutic drugs to the tissue of the subject or the sampling of biofluids for the purposes of diagnosis. In one embodiment of the invention, a rigid structure has defined ingrowth features on a surface intended to contact tissue of the subject and defined passage features which provide a fluid path from the surface intended to contact tissue to another surface. The dimensions of these defined features vary based on the particular application, as the ingrowth features are of a dimension and spacing to promote ingrowth of the surrounding tissue, and the passage features are of a dimension to inhibit the passage through the structure of cells from the surrounding tissue.

### RELATED APPLICATIONS

This application claims priority under 35 U.S.C. §119(e) to U.S. Provisional Application 60/519,060 filed on Nov. 10, 2003.

### BACKGROUND OF THE INVENTION

1. Field of the Invention

This invention relates to structures allowing fluid passage between the interior lumen of an implanted device and the surrounding tissue. These structures may be part of a drug delivery system/device or a biofluid sampling system/device intended for use within a mammalian body. More particularly, embodiments of the invention provide for structures that promote surrounding tissue ingrowth onto the outer aspect of the structure while preventing cellular ingrowth through the structure into the lumen of the device.

2. Description of the Related Art

An important yet still unmet need in the medical community is for implanted devices which provide ready access to bodily fluids over extended periods of time, e.g. days, weeks or months. These devices may be used, for example, in parenteral drug administration or in biofluid sampling, such as for the purpose of glucose monitoring. One cause of shortened useful lifetimes of implanted devices is the encapsulation of such devices by surrounding immune response cells and/or scar tissue which inhibit interaction between the device and normal vascularized tissue. One method of extending the useful lifetimes of such devices is to minimize the encapsulation response through the use of surface features on the implanted devices, while simultaneously promoting vascular ingrowth.

However, to provide for efficient fluid transfer, such structures must also limit the ingrowth of tissues into fluid passages. Such ingrowth occludes the fluid path and potentially may invade device lumenal space. To accomplish this task, several approaches based upon a multiplicity of pore sizes have been proposed. In general, the outer aspects of these structures employ a loose network or multi micron construct permitting surrounding tissue ingrowth. The inner aspect is typically a fine mesh or porous network having dimensions such that cellular ingrowth is physically constrained. To date, these approaches require multiple layers or laminated constructions or require underlying physical support structures to preserve device lumenal space.

For example, Gowda and McNicols (U.S. Pat. No. 6,459,917) teach the use of a filtration membrane having micro architecture to promote neovascularization. In order to prevent cells from entering the collection reservoir, an ultrafiltration membrane having a pore size of less than 1.0 μm is laminated to the filtration membrane. Such a multilayered structure requires multiple assembly steps. In addition, such a flexible membrane may require still additional structures to provide additional mechanical support and may be subject to delamination and therefore failure in operation.

In a similar vein, Brauker et al., U.S. Pat. No. 5,741,330 and Shults et al., U.S. Pat. No. 6,001,067 use membrane-like structures in bilayers to promote tissue ingrowth while precluding cellular migration. As above, these structures are laminate in nature, requiring support means and may be subject to delamination.

Joseph and Torjman (U.S. Pat. No. 6,471,689) describe a drug delivery catheter system having a support structure between the lumen of the catheter having a plurality of holes for drug delivery from the lumen and into the mammal. A capillary interface is disposed about the support structure and includes an outer portion to facilitate ingrowth of vascular tissue and an inner portion adapted to inhibit ingrowth of vascular tissue while permitting the flow of drugs from the support structure out through the capillary interface. This system requires an underlying support having a plurality of holes capable of withstanding mechanical load from surrounding tissue upon the membrane, i.e. the support structure, distinct from the structure(s) providing the capillary interface. In addition to requiring an underlying support, such a system requires the manufacture and assembly of multiple components.

Therefore, there remains a need for a single structure that provides a simple, efficient structure to provide fluid transfer between a lumen or other form of reservoir and the surrounding tissue of a mammal while providing for neovascularization while simultaneously limiting surrounding cell ingrowth.

### SUMMARY OF CERTAIN INVENTIVE ASPECTS

In an embodiment of the invention, there is a device for implantation within a subject, comprising rigid structure having at least first and second surfaces, wherein at least the first surface has a predefined pattern of ingrowth features configured to promote tissue ingrowth; an interior lumenal space at least partially defined by the second surface of the rigid structure; and a predefined pattern of passages extending between the first surface and the second surface of the rigid structure, such that the interior lumenal space can be placed in fluid communication with tissue of the subject.

In another embodiment of the invention, there is a rigid structure for implantation within a subject, comprising a first surface, said first surface comprising a predefined pattern of ingrowth features extending outward from said first surface, configured to contact tissue within the subject and promote tissue ingrowth; a second surface; and a predefined pattern of passages extending from the first surface to the second surface, wherein the passages are of sufficiently small dimension to preclude mammalian cellular passage via the passages.

In another embodiment of the invention, there is a method of manufacturing a rigid structure for use in a device implantable within the tissue of a subject, comprising selectively removing material from a structure in order to create a predefined pattern of passage features having at least one dimension sufficiently small to preclude mammillian cellular passage, and selectively removing material from a first surface of the structure in order to create a predefined pattern of ingrowth features configured to promote tissue ingrowth.

In another embodiment of the invention, there is a method of manufacturing a rigid structure for use in a device implantable within the tissue of a subject, comprising selectively depositing material in order to create a structure comprising a predefined pattern of passage features and ingrowth features, wherein the passage features have at least one dimension sufficiently small to preclude mammillian cellular passage, and wherein the ingrowth features are configured to promote tissue ingrowth.

### BRIEF DESCRIPTION OF THE DRAWINGS

FIG. 1 is a cross-sectional side view of an embodiment of the invention.

FIG. 2 is an illustration of an embodiment of the invention having pillar-like ingrowth features.

FIG. 3 is an illustration of an embodiment of the invention having troughs.

FIG. 4 is an illustration of an embodiment of the invention having features which are effectively “random” in composition over the region shown.

FIG. 5 is a cross-sectional side view of an embodiment of the invention having several different forms of micron scale ingrowth features.

FIG. 6 is an illustration of an embodiment of the invention comprising a device insertable in the tissue of a subject.

### DETAILED DESCRIPTION OF CERTAIN INVENTIVE EMBODIMENTS

The following description presents certain specific embodiments of the invention. However, the invention may be embodied in a multitude of different ways as defined and covered by the claims. In this description, reference is made to the drawings wherein like parts are designated with like numerals throughout.

As used herein, the term biofluids refers to fluids found in extracellular environments, e.g. interstitial fluid, cerebrospinal fluid, throughout the body of the subject which may contain a variety of materials, including but not limited to, proteins, hormones, nutrients, electrolytes, catabolic products, or introduced foreign substances.

A rigid structure is one comprising those fabricated materials effectively solid and rigid enough to form an essentially unsupported side wall or side wall portion of an implanted device.

As used in this specification, tissue substance transfer refers to the transfer of a substance or material either into or out of the tissue of the subject. Tissue substance transfer may refer, for example, to the transfer of biofluids from the tissue of a subject to a device implanted either completely or percutaneously within the tissue of the subject. Tissue substance transfer may also refer to the transfer of a substance or material, such as therapeutic drugs, to the tissue of a subject from a device implanted either completely or percutaneously within said tissue.

The invention generally relates to novel microarchitecture structures, and their use with implanted drug delivery and biofluid sampling devices. Certain advantageous embodiments of the invention relate to rigid structures, as opposed to flexible porous polymers, having defined micron scale features to promote tissue ingrowth and having defined micron to submicron scale passage features to permit fluid transfer between the outside and inside of a device.

In one embodiment, the micron scale ingrowth features and the submicron scale passage features providing a fluid path through the rigid structure may be constructed from a contiguous solid material without division or layering between these two features. Certain embodiments of the invention provide multiple advantages over other biointerface structures composed of membranes and/or polymers for many reasons. An embodiment of the invention advantageously avoids possible device failure due to delamination between membrane regions. This embodiment of the invention is structurally defined and rigid, advantageously not requiring underlying support structure. This embodiment also advantageously provides for simplified device design and manufacture. The manufacturing and materials used in an embodiment of the invention advantageously allow for the addition of additional features if desired, such as a surface coating of additional biocompatible materials.

Certain embodiments of the invention may be fabricated using standard semiconductor processing techniques and materials. Such techniques allow for precise definition of surface features in a high volume, and highly reproducible fashion at a variety of dimensions, e.g. micron to centimeter.

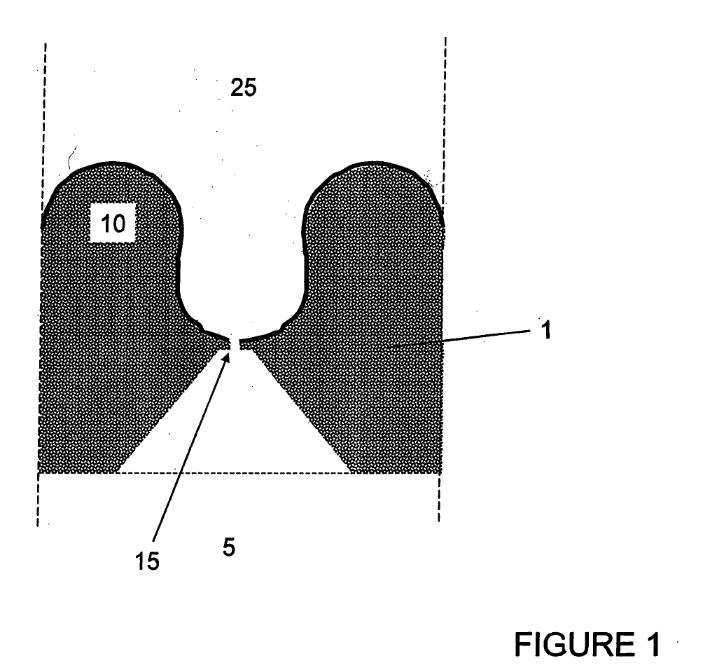

In one embodiment of the invention, a rigid structure possesses at least one surface having a plurality of micron scale ingrowth features that are intended to contact the tissue of the mammalian implant subject. As a contiguous extension of the surface of these micron scale ingrowth features, there is second set of passage features, sub-micron to micron scale in at least one dimension, which provide a fluid path to at least one other surface of the structure. FIG. 1 illustrates a cross sectional view of a general rendition of such a structure. Such structures may be effectively planar in the overall shape or may be constructed as curvilinear surfaces or other three dimensional forms having the micron and sub-micron scale ingrowth features upon the outer surface, and the sub-micron passage features providing a fluid path from the outer surface through the structure to another surface of the structure.

As shown in FIG. 1, the structure 1 has micron scale ingrowth features 10 upon the outer aspect of the structure which are in contact with surrounding tissue 25. The structure also has a plurality of passage features 15 that aresub-micron to micron in scale allowing fluid passage between an interior lumenal region 5 of a device possessing said structure and the surrounding tissue 25. Other embodiments of structures are readily conceivable and FIG. 1 is not intended to limit the scope of the invention.

In embodiments of the invention, micron scale ingrowth features on the outer aspect of a structure are intended to promote tissue ingrowth including possible neovascularization. These ingrowth features in general reflect the dimensionality of the surrounding cells and tissues. Accordingly, ingrowth features may range in scale from micron to the multimicron. Ingrowth feature sizes in the range of 1 to 100 microns in at least one of three possible dimensions are generally considered appropriate for soft tissue applications. For other tissues and/or applications, other dimensions, such as 100 μm to 400 μm for bone, may be more appropriate. In addition, nanoscale subfeatures and/or molecular entities may be added to the micron scale ingrowth features to improve the overall performance of the micron scale topology.

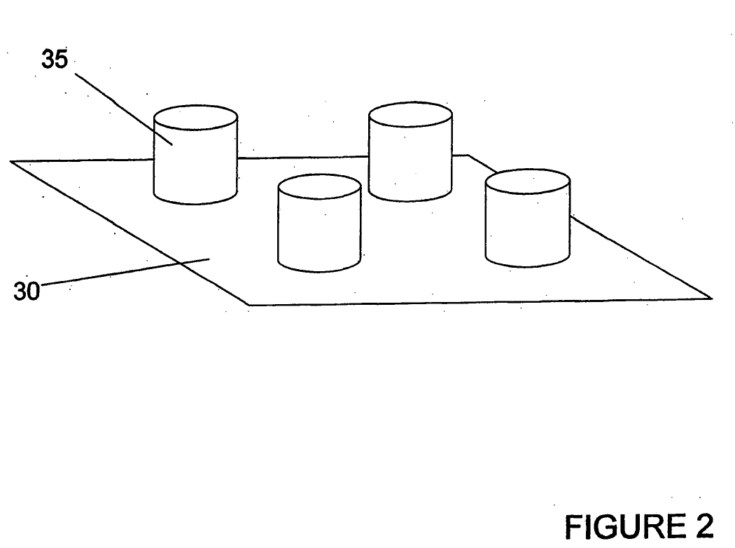

In various embodiments of the invention, the micron scale ingrowth features may be in the form of grooves, channels, pits or other surface topologies to promote surrounding tissue acceptance. FIGS. 2, 3, and 4 are representations of such topologies which may be employed.

FIG. 2 illustrates an embodiment of the invention having post-like micron scale ingrowth features. The base structure 30 has a plurality of post or pillar shape ingrowth features 35 arrayed upon the outer surface. In one embodiment of the invention, such pillars are preferably between 1 micron and 50 microns in diameter and have center to center dimensions allowing spacing between adjacent pillars of greater than 2 microns and less than 1000 microns. Heights of such pillars may be between 2 microns and 500 microns and may vary from post to post. In various embodiments of the invention, the size and arrangement of such pillars about the surface of the structure may adopt a variety of forms and dimensions and should not be limited to the structures and arrangements shown in FIG. 2.

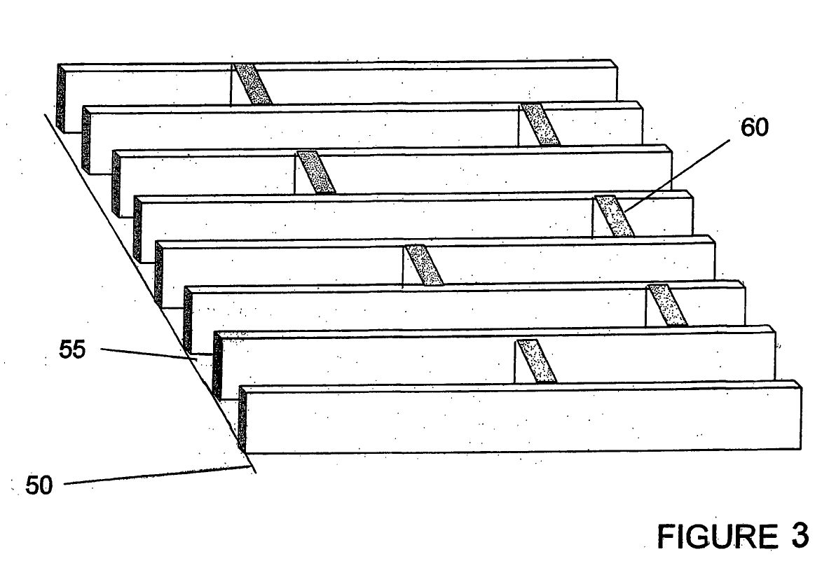

FIG. 3 represents an alternative embodiment of the invention having micron scale ingrowth features in the form of troughs 55 arrayed upon the surface of the structure 50. Such troughs preferably range from 2 microns to 1000 microns in length and from 2 microns to 500 microns in width. Heights of such structures preferably range from 2 microns to 500 microns. Also shown in FIG. 3 are cross members 60 forming the ends of the troughs upon the structure. Such cross members may replicate the height of the surrounding troughs, as shown, or adopt dimensions either greater or lesser than the walls of the troughs. The width of the trough walls may vary from 2 microns to 100 microns. The preferable dimensions of the features will vary depending on the application.

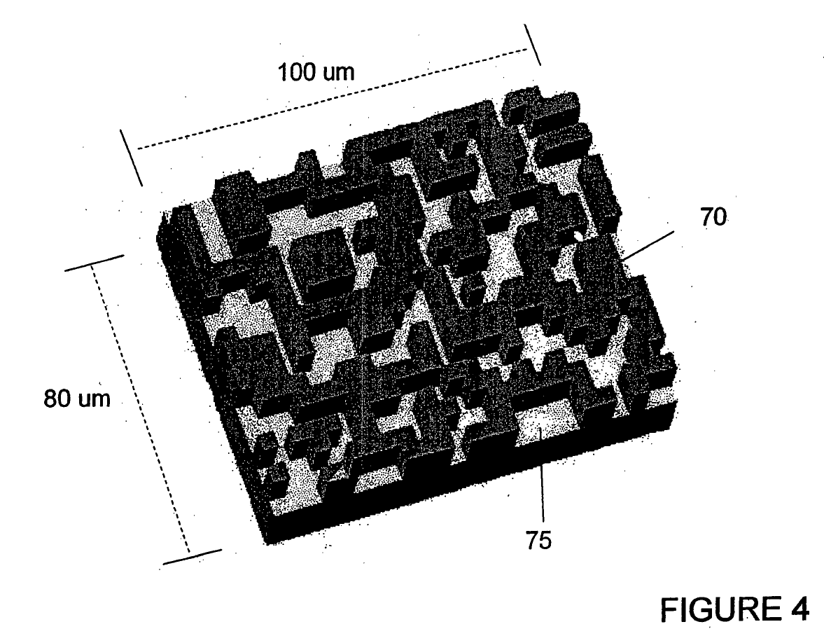

FIG. 4 illustrates one embodiment of the invention having non-repetitive or effectively random patterns of micron scale ingrowth features 70 which define troughs 75. A desirable element of such patterns is that repetition of ingrowth features, if occurring, is on a dimension greater than that traversable by a single mammalian cell. Therefore, in one embodiment of the structure of this invention employing such random micron scale ingrowth features, a pattern of such features extends at least 50 microns prior to its repetition. While in one embodiment of the invention the pattern is described as being effectively random, and while in further embodiments of the invention no repetition of the pattern may occur, the pattern of ingrowth features may nevertheless be a defined pattern.

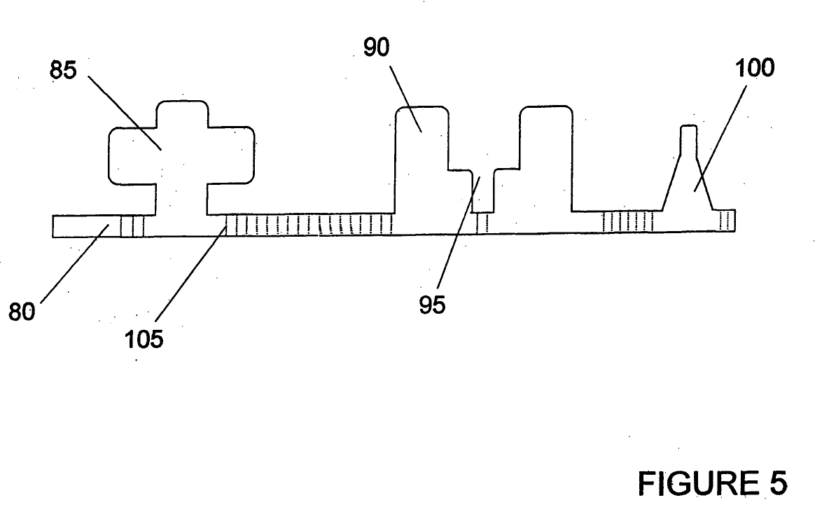

Additional embodiments of the invention may make use of layered ingrowth features, such as stepped or overhanging ingrowth features or other combinations of ingrowth features. Additional embodiments of the invention may also make use of ingrowth features possessing rounded edges, corners or other non-rectilinear dimensions. A multitude of other variations in the shape and combinations of ingrowth features are conceivable and within the scope of this invention. FIG. 5 illustrates an embodiment of the invention making use of a variety of ingrowth feature shapes.

Still with reference to FIG. 5, alternative embodiments of the invention may provide ingrowth features upon the surface 80 such as holes or connections having dimensions suitable for one or more cells to penetrate in whole or in part. The cross-sectional shape of ingrowth figures may vary at different distances from the surface 80, creating overhang or stepped features, as seen on ingrowth FIG. 85, or tapering features, as seen on ingrowth feature 100. In an alternative embodiment of the invention, ingrowth features 90 may include cavity features 95 which preclude ingress of surrounding tissue and have at their inner aspect micron or submicron scale passage features 105. Likewise, non-planar forms for the overall structure of embodiments of the invention are conceivable, including forms for the structure which adopt ovoid, toroid or other shapes. Combinations of one or more micron scale ingrowth features may be employed on the structure and are within the scope of the invention.

The variety of ingrowth features depicted in FIG. 5 are representative of the precision which can be obtained through the use of materials which are both biocompatible and suitable for use in semiconductor processing techniques. The ingrowth features can be defined with a high degree of precision, enabling the creation of the various ingrowth features depicted therein as well as a multitude of alternate shapes. In addition, it is possible to utilize these processing techniques to generate structures which are contiguous in their design, adding to the rigidity of the device and decreasing the likelihood of device failure due to delamination.

In one embodiment of the invention, a plurality of passage features 105 are provided between the ingrowth features, as illustrated in FIG. 5. These passage features provide a fluid path between one or more non-tissue contacting surfaces of the structure with one or more surfaces having micron scale ingrowth features which are intended for contact with tissue. In a preferred embodiment of the invention, these passage features are substantially perpendicular to the surface upon which micron scale ingrowth features are present. Typically, these passage features have at least one cross sectional dimension generally in the range of 1 micron to 10 nanometers at at least one point along the fluidic path within the structure.

In various embodiments of the invention, the passage features may constitute a variety of shapes and dimensions while traversing from the inner aspect to outer aspect of the device. In addition, one or more passage features may be located in the space between any adjacent micron scale features, as illustrated in FIG. 5. These passage features have at least one cross-sectional dimension generally in the range of 1 micron to 10 nanometers at least one point along the fluidic path within the structure. In further embodiments of the invention, one or more passage features may converge to form larger passages. Such embodiments may provide advantages for adjustment of fluid delivery rates and pressures.

In an embodiment of the invention, one function of these passage features is to provide a fluid path. In a further embodiment of the invention, these passage features may be used to provide a path for fluid transfer from the interior of the device to the surrounding interstitial space. In alternative further embodiments, the passage features provide a path for fluid transfer from the surrounding tissue into a lumenal space of a device having the structure of this invention.

In various embodiments of the invention, such fluids may be employed for therapeutic delivery of drugs, agents or other substances from a device into the surrounding tissue. Alternative embodiments of the invention may be used in the collection or sampling of biofluids for specific analytes. Alternative embodiments may be used in the delivery of nutrients, proteins or other biological substances to cells, organelles or other living entities enclosed within a device utilizing embodiments of the invention. Embodiments of the invention advantageously provide the ability to combine small pore size (submicron or nanometer scale pores) with larger micron scale surface topology, and represent a novel advancement in the use of rigid structures for devices implanted within the body and offers a variety of applications both for drug delivery and diagnostic sampling.

By utilizing semiconductor processing techniques in the manufacture of embodiments of the invention, a far greater control over the behavior of the embodiment can be obtained. In embodiments of the invention which serve as tissue substance transfer devices, the increased amount of control over the fluid flow through a rigid structure permits greater control over the performance of the device. The semiconductor processing techniques utilized in the fabrication of certain embodiments also enable the creation of passages having greater consistency in shape and size than is possible in devices employing polymer membranes.

In practice, the length of such passages having micron or submicron cross sectional dimensions is set by the limits of current etching or other pore forming technologies. In general, such passages are considered to be between 1 micron and 20 microns in length having aspect ratios of generally less than 20 to 1. However, the scope of this invention shall be in accordance with technical advancement and includes alternate methods of forming such passages including but not limited to, removal of select regions of material, e.g. etching techniques, or addition of materials having appropriate dimension, e.g. growing a portion or all of the structure of this invention, .e.g. sol-gel techniques, or some combination of the two.

In certain embodiments of the invention, the passage diameter, if of a sufficiently narrow aspect, e.g. <250 nm, may also serve as a final barrier preventing an infection route to bacteria from the interior of the device into the surrounding tissues. This function may also be served in alternate embodiments of the invention by a second structure, e.g. a microporous filter, frit or membrane, placed in substantial contact with the inner aspect of the structure or by a filter placed elsewhere in the fluidic path within the device.

As various embodiments of the invention include both micron and submicron scale features, suitable materials for such construction must be utilized. In addition, because various embodiments of the invention require that the device be implanted either completely or percutaneously, the biocompatibility of materials used is a concern. Suitable materials for use in the manufacture of these embodiments include, but are not limited to, those materials suitable for MEMS (MicroElectroMechanical Systems) fabrication which are also suitable for biocompatibility. These include, but are not limited to, silicon, silicon oxide, silicon nitride, silicon carbide, titanium, and the photoresist polymer SU-8 (MicroChem Corporation, Newton, Mass.; G. Kozar. et al., “Evaluation of MEMS Materials of Construction for Implantable Devices.” Biomaterials 23 (2002) 2737-2750). In addition, other materials, such as solid polymers, ceramics, glasses, other metals or metal alloys, e.g. platinum, indium or platinum-indium alloys, as well as heterogeneous or composite materials, may be utilized in construction of all or parts of the elements of the structures of this invention.

Construction of the various embodiments of the invention using these materials may be accomplished using tools and processes well known to those skilled in the art of micromachining or semiconductor fabrication. These tools and processes include, but are not limited to, chemical etching, deep reactive ion etching, laser etching, and electrochemical deposition. In addition, other tools or processes may be suitable for construction of these structures and the scope of this invention is not limited to any one particular process, material or fabrication method.

An embodiment of the invention may be formed by the selective removal of material via, for example, an etching or micromachining method. Alternately, an embodiment may be constructed by the selective deposition of material via a deposition method. Alternative methods of manufacture may comprise a combination of selective deposition and removal of materials, including the deposition and removal of sacrificial layers. It will be understood that the deposition and removal of material need not occur in a particular order, and that a multitude of satisfactory combinations of particular deposition and removal methods may be utilized in order to manufacture embodiments of the invention.

Semiconductor processing techniques enable the manufacture of a rigid structure having a predefined pattern of ingrowth and passage features. Therefore, embodiments of the invention may be constructed such that the manufacturer is aware of the exact topology of the structures created using these techniques. Such precision cannot be achieved with the use of polymer membranes. Modifications to these topologies can be made so as to advantageously optimize the behavior of a device depending on the particular tissue with which the surface is intended to come into contact. As illustrated in FIG. 5, these modifications may extend well beyond optimizing the height, width and length of ingrowth features and the distance between those features. The use of semiconductor processing techniques and suitable material permits the creation of ingrowth features having very precisely designed shapes.

In one preferred embodiment of the invention, titanium is employed as the material comprising a substantial portion of the structure. Titanium, along with its associated derivatives such as titanium oxide, is a material well known for its biocompatibility and has been extensively utilized in medical implants, catheters and related devices. The material is cheap, non-brittle, and strong, in addition to its known biocompatibility. Titanium may compose the entirety of the structure, i.e. the structure being a solid, homogenous assembly fabricated entirely from titanium, or titanium may be plated onto an underlying material, e.g. silicon, or otherwise be employed as a component of the structure.

The manufacture of such titanium, or titanium-including, structures may be done by a variety of methods, including but not limited to, electro-deposition; physical vapor deposition; vacuum arc deposition, chemical deposition; micro machining or etching. An embodiment of the invention may be either effectively homogenous in composition, i.e. primarily titanium or titanium alloy, having the appropriate dimensions, shapes or surfaces at the nanometer or micrometer scale necessary for biocompatibility and device performance (such as therapeutic agent delivery or the passage of biofluids for the purpose of physiological monitoring). Alternative embodiments may be composed entirely or in regions, layers or other heterogeneous forms of one or materials.

In alternate embodiments of the invention, additional layers of materials may be added to either the outer aspect or inner aspect of the structure. Such layers may include, but are not limited to gels, fibrous polymers, polymeric meshes, metallic micron or nanoscale materials as well as microporous frits. These materials may be employed for a variety of possible functions, including but not limited to, enhancing tissue ingrowth, drug delivery coatings, anti-inflammatory drug release, or providing bacterial-static activities.

Embodiments of the invention have a wide area of application in the areas of diagnostics and drug delivery. Individual applications may be tailored to fit the site of implantation, e.g. organ as compared to subcutaneous as compared to intraperitoneal, etc., as well as delivery/sampling needs, e.g. volumes required per unit time, as well as comfort, e.g. multiple sub-millimeter scale devices as opposed to unitary multimillimeter scale devices. In addition, devices employing one or more structures of this invention may be wholly implanted or percutaneous in nature.

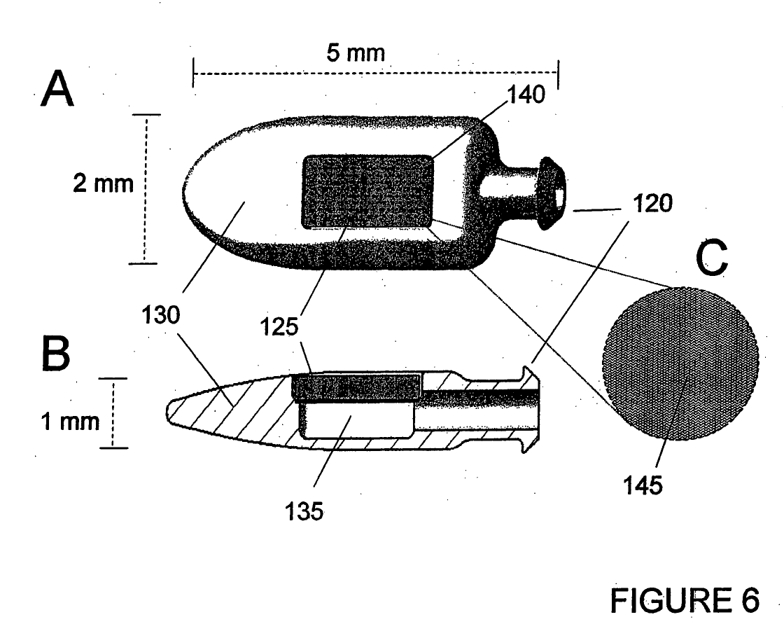

FIG. 6 illustrates a portion of a conceptual percutaneous drug delivery device. FIG. 6A shows a top view of the device. FIG. 6B shows a cut-away side view of the device approximately through the midline of the device. FIG. 6C shows an expanded view of the top surface of a rigid structure 125 placed into the body of a device 130. The device 130 may be placed in fluid communication with a further device, such as a catheter, via a collared aperture 120, in order to enable deeper implantation. As shown in both 6A and 6B, the body of the device 130 has the rigid structure 125 mounted. The rigid structure 125 provides a fluid path from the lumenal space 135 of the device to the outer aspects of the device. Representations of the plurality of submicron passage features 140 are shown evenly arrayed on the rigid structure 125. Such representations are not to the scale of the drawing. Likewise, 6C illustrates micron scale texturing 145, e.g. curvilinear troughs on the upper surface of the structure, again not to the scale of the drawing.

Due to the rigidity of rigid structure 125, an embodiment of the invention as depicted in FIG. 6 may be constructed without the need for additional support for the rigid structure 125, unlike similar devices which employ polymer membranes. Due to the selection of materials and processing techniques, the device is capable of being inserted into tissue without the rigid structure 125 experiencing substantial deformation due to the pressure exerted on the structure by the surrounding tissue. As discussed above, the increased rigidity also leads to simplified device manufacture, as the need for membrane support increases the complexity, and therefore the cost and reliability, of the device.

While embodiments of the invention may be constructed such that a rigid structure employed in the design of the device is made from a contiguous piece of, for example, biocompatible material such as titanium or its derivatives, an additional layer can also be utilized in providing additional functionality, such as that discussed previously. Due to the rigidity of the structure and the resulting lack of substantial deformation when pressure is applied to the structure by the surrounding tissue after insertion, less stress is placed on the interface between the structure and any additional layers. The likelihood of device failure due to delamination is therefore advantageously reduced.

In select embodiments of the invention, devices may be constructed that are in general shape and form suitable for drug delivery as well as providing access to biofluids for diagnostic sampling, e.g. for the detection of one or more analytes. An embodiment of such a percutaneous device is described in U.S. patent application Ser. No. 10/032,765, now U.S. Publication Number 2004-0004403 A1, “Gateway Platform for Biological Monitoring and Delivery of Therapeutic Compounds” which is incorporated by reference in its entirety herein. It is understood that applications for drug delivery will contain elements possibly differing from those for biofluid sampling, e.g. pumps and reservoirs as compared to sensor elements.

In alternate embodiments of the invention, a device is fully contained within the tissues of the subject, e.g. in the form of a subcutaneously implanted pill. Alternatively, embodiments of the invention may comprise catheters, probes or other devices for delivery of fluids and possible sampling of biofluids or components of the biofluids. These devices may also serve to deliver nanoagents or other nano-scale constructs designed for either local activity within surrounding tissue or for more systemic activities.

In still other embodiments of the invention, a device may house introduced systems or devices, e.g. a drug delivery system or sensor apparatus, within a lumenal space of the device and therefore provide a fluid path allowing these introduced systems and devices to interact with the host tissue and bodily fluids while being segregated from the encapsulation response possibly ensuing if these introduced devices were introduced in the absence of an embodiment of the invention. In further embodiments of the invention, the device is percutaneous in nature and said introduced systems and introduced devices are insertable down a catheter-like tubing to the lumenal space within the device. Such insertions may permit the use of removable and replaceable systems and devices within these embodiments.

As noted above, some embodiments of the invention may be devices suitable in general form for both drug delivery and for analyte detection. In alternate embodiments of the invention, a device is constructed solely for the purpose of sampling biofluids for diagnostic purposes. In-yet other embodiments of the invention, a device has one or more living cells present within the lumen of the device. Such cells may be genetically engineered to serve as living sensor systems, e.g. upon sensing a particular analyte in biofluid such as a toxin, the cell may be engineered to respond with expression of a green fluorescent protein signaling the presence of the toxin. In other embodiments of the invention the cells may either be unaltered or enhanced and designed to respond to hormonal or nutrient signals within the biofluid. An example of such a response might be pancreatic islet cells responding to glucose levels in the biofluid and secreting insulin in response. Such examples are provided as illustrations and are not intended to limit the scope of the invention.

While the above detailed description has shown, described and pointed out the fundamental novel features of the invention as applied to various embodiments, it will be understood that various omissions and substitutions and changes in the form and details of the system illustrated may be made by those skilled in the art, without departing from the intent of the invention. The foregoing description details certain embodiments of the invention. It will be appreciated, however, that no matter how detailed the foregoing appears, the invention may be embodied in other specific forms without departing from its spirit or essential characteristics. The described embodiment is to be considered in all respects only as illustrative and not restrictive and the scope of the invention is, therefore, indicated by the appended claims rather than by the foregoing description. All changes which come within the meaning and range of equivalency of the claims are to be embraced within their scope.

1. A device for implantation within a subject, comprising:
a rigid structure having at least first and second surfaces, wherein at least the first surface has a predefined pattern of ingrowth features configured to promote tissue ingrowth;
an interior lumenal space at least partially defined by the second surface of the rigid structure; and
a predefined pattern of passages extending between the first surface and the second surface of the rigid structure, such that the interior lumenal space can be placed in fluid communication with tissue of the subject.

2. The device of claim 1, wherein the structure has sufficient rigidity to resist significant deformation due to the pressure placed on said structure by the surrounding tissue without underlying physical support.

3. The device of claim 2, wherein biocompatible material is employed as a component of the rigid structure.

4. The device of claim 3, wherein the biocompatible material comprises titanium, titanium oxide, or a titanium alloy.

5. The device of claim 3, wherein the rigid structure is biocompatible.

6. The device of claim 3, wherein at least the first surface of the rigid structure is coated with a layer of biocompatible material.

7. The device of claim 6, wherein the biocompatible material comprises titanium, titanium oxide, or a titanium alloy.

8. The device of claim 1, additionally comprising at least one additional layer of material in contact with a surface of the rigid material.

9. The device of claim 1, wherein the lumenal space is configured for insertion of an introduced device.

10. The device of claim 9, wherein the introduced device comprises a sensor apparatus.

11. The device of claim 9, wherein the introduced device comprises a drug delivery system.

12. A rigid structure for implantation within a subject, comprising:
a first surface, said first surface comprising a predefined pattern of ingrowth features extending outward from said first surface, configured to contact tissue within the subject and promote tissue ingrowth;
a second surface; and
a predefined pattern of passages extending from the first surface to the second surface, wherein the passages are of sufficiently small dimension to preclude mammalian cellular passage via the passages.

13. The rigid structure of claim 12, wherein the structure has sufficient rigidity to resist significant deformation due to the pressure placed on said structure by the surrounding tissue without underlying physical support.

14. The rigid structure of claim 12, wherein the rigid structure is at least partially composed of biocompatible material.

15. The rigid structure of claim 14, wherein the biocompatible material comprises titanium, titanium oxide, or a titanium alloy.

16. The rigid structure of claim 14, wherein the rigid structure is entirely composed of contiguous biocompatible material.

17. The rigid structure of claim 14, wherein at least the first surface of the rigid structure is coated with a layer of biocompatible material.

18. The rigid structure of claim 17, wherein the biocompatible material comprises titanium, titanium oxide, or a titanium alloy.

19. The rigid structure of claim 12, additionally comprising at least one additional layer of material in contact with a surface of the rigid material.

20. The rigid structure of claim 12, wherein the predefined pattern of ingrowth features is determined based partially on the particular type of tissue with which the rigid structure will be in contact.

21. A method of manufacturing a rigid structure for use in a device implantable within the tissue of a subject, comprising:
selectively removing material from a structure in order to create a predefined pattern of passage features having at least one dimension sufficiently small to preclude mammillian cellular passage; and
selectively removing material from a first surface of the structure in order to create a predefined pattern of ingrowth features configured to promote tissue ingrowth.

22. The method of claim 21, wherein the structure is formed at least partially of biocompatible material.

23. The method of claim 22, wherein the biocompatible material is titanium or a titanium derivative.

24. The method of claim 21, wherein the structure is formed entirely of biocompatible material.

25. The method of claim 21, additionally comprising depositing a layer of biocompatible material on at least the first surface of the structure.

26. The method of claim 24, wherein the biocompatible material is titanium or a titanium derivative.

27. The method of claim 21, wherein the material is selectively removed via laser etching.

28. The method of claim 21, wherein the material is selectively removed via chemical etching.

29. The method of claim 21, wherein the material is selectively removed via deep reactive ion etching.

30. The method of claim 21, wherein the material is selectively removed via micromachining.

31. The method of claim 21, additionally comprising a first step of constructing the structure by deposition of various layers of material.

32. The method of claim 31, wherein the various layers of material are deposited in defined patterns.

33. A method of manufacturing a rigid structure for use in a device implantable within the tissue of a subject, comprising selectively depositing material in order to create a structure comprising a predefined pattern of passage features and ingrowth features, wherein the passage features have at least one dimension sufficiently small to preclude mammillian cellular passage, and wherein the ingrowth features are configured to promote tissue ingrowth.

34. The method of claim 33, wherein at least a portion of the material selectively deposited is sacrificial material.

35. The method of claim 33, further comprising selectively removing a portion of the deposited material.

36. The method of claim 35, additionally comprising the deposition of sacrificial material, and wherein at least a portion of the material selectively removed is said sacrificial material.

37. The method of claim 33, wherein the material is selectively deposited via electro-deposition.

38. The method of claim 33, wherein the material is selectively deposited via physical vapor deposition.

39. The method of claim 33, wherein the material is selectively deposited via vacuum arc deposition.

40. The method of claim 33, wherein the material is selectively deposited via chemical deposition.

41. The method of claim 33, wherein the at least a portion of the selectively deposited material is titanium or a titanium derivative.
